(19) United States
(12) Patent Application Publication
(10) Pub. No.: US 2026/0286262 A1
(43) Pub. Date: Sep. 24, 2026

(54) DEVICE FOR THE PREPARATION OF VASCULARIZED 3D CELL CULTURE SYSTEMS AND USES THEREOF

(71) Applicant: VALORISATION HSJ, LIMITED PARTNERSHIP, Québec, Québec (CA)

(72) Inventors: Noël RAYNAL, Mont-Royal, Quebec (CA); Arman JAFARI, Montreal, Quebec (CA); Houman SAVOJI, Laval, Quebec (CA)

(21) Appl. No.: 19/474,546

(22) PCT Filed: Apr. 11, 2024
(86) PCT No.: PCT/CA2024/050457

§ 371 Date: Oct. 10, 2025

Related U.S. Application Data
(60) Provisional application No. 63/495,574, filed on Apr. 12, 2023.

(51) Int. Cl. C12M 3/00; C12M 1/12; C12N 5/00; C12N 5/071; C12N 11/04

(52) U.S. Cl. CPC C12M 21/08; C12M 25/14; C12N 5/0062; C12N 5/069; C12N 11/04; C12N 2533/54

(57) ABSTRACT

Cell culture is generally performed in two-dimensional (2D) petri dishes or tissue culture flasks. These growing conditions provide an artificial microenvironment that interferes with the natural cell behavior, phenotype and drug responses. The present disclosure provides a growth microenvironment that alleviates at least some of these drawbacks. The present application relates to a 3D-cell culture preparation device comprising a pronged component comprising a plurality of outwards prongs, the plurality of prongs being spatially distributed in accordance with a target artificial vasculature; and a receiver component defining a 3D-cell culture cavity when positioned onto the pronged component, the pronged component letting the plurality of prongs into the 3D-cell culture cavity, wherein the device is configured to receive a polymerizable liquid in the 3D-cell culture cavity.

### CROSS REFERENCE TO RELATED APPLICATIONS

The present application claims the benefit of U.S. provisional patent application Ser. No. 63/495,574, filed on Apr. 12, 2023, which is incorporated herein by reference in their entirety.

### TECHNICAL FIELD

The present disclosure generally relates to the field of cell culture, and more particularly to devices and systems the culture of cells.

### BACKGROUND ART

Cell culture is generally performed in two-dimensional (2D) petri dishes or tissue culture flasks. These growing conditions provide an artificial microenvironment that interferes with the natural cell behavior, phenotype and drug responses. Unrealistic 2D conditions translate into the poor performance of many preclinical models and as a result, it is not uncommon for drugs tested in clinical trials to never be approved due to lack of efficacy.

There is thus a need for improved 3D cell culture devices and systems.

The present disclosure provides a growth microenvironment that alleviates at least some of these drawbacks.

The present description refers to a number of documents, the content of which is herein incorporated by reference in their entirety.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

In a first aspect, the techniques described herein relates to a 3D-cell culture preparation device. The device comprises a prong component and a receiver component. The pronged component is a plurality of outward prongs. The plurality of prongs is spatially distributed in accordance with the target artificial vasculature. The receiver component defines a 3D-cell culture cavity when positioned onto the pronged component. The plurality of prongs are protruding into the 3D-cell culture cavity. The device is configured to receive a liquid, such as a polymerizable hydrogel, in the 3D-cell culture cavity.

Optionally, the techniques described herein may further relate to a 3D-cell culture preparation device where the prongs protrude through the 3D-cell culture cavity once the receiver component is positioned onto the pronged component.

Optionally, in addition or alternatively, the 3D-cell culture preparation device may further include an opening defined to receive the liquid toward the 3D-cell culture cavity. The 3D-cell culture cavity may thereby be stanched to the liquid when the receiver component is operably positioned over the pronged component.

Optionally, in addition or alternatively, the volume of the 3D-cell culture cavity may be defined to receive between 0.5 cubic centimeter (cm³) and 1.5 cubic centimeters of the liquid.

Optionally, in addition or alternatively, the receiver component may comprise a hole in a base thereof for each of the plurality of prongs. In such a case, at least one additional hole in the base of the receiver component may further be left unoccupied by the plurality of prongs such as to allow the liquid to be inserted into the 3D-cell culture cavity and/or to facilitate the extraction of the polymerized liquid (e.g., hydrogel) from the scaffold.

Optionally, in addition or alternatively, the 3D-cell culture preparation device may further comprise a holder portion configured to facilitate separation of the receiver component and pronged component.

Optionally, in addition or alternatively, the spatial distribution leaves at least about 0.1 or 0.2 mm, for example at least about 0.4 mm, between each of the plurality of prongs and the plurality of prongs occupies between about 5 to about 50%, about 5 to about 40%, about 10 to about 30% or about 10% to about 25% of a culture volume of the 3D-cell-culture cavity. The culture volume is defined by a volume of liquid received in the 3D-cell-culture cavity. Optionally, in addition or alternatively, the spatial distribution leaves about 0.4 mm to about 0.8 mm between each of the plurality of prongs, for example about 0.5 mm to about 0.7 mm, about 0.55 mm to about 0.65 mm, about 0.56 mm to about 0.64 mm, about 0.57 mm to about 0.63 mm, about 0.58 mm to about 0.62 mm, about 0.59 mm to about 0.61 mm, or about 0.6 mm.

Optionally, in addition or alternatively, the prongs from the plurality of prongs may be shaped as one or more of a cylinder, a polygonal base prism, an elliptical prism, a histologically-inspired prism and an enamel-inspired prism.

In a second aspect, the techniques described herein relates to a method for preparing a 3D-cell culture with improved nutrient diffusion, oxygen supply and/or waste removal from cells embedded therein. A polymerizable liquid (e.g., hydrogel) is poured into a mold. The mold comprises a pronged component and a receiver component. The pronged component comprises a plurality of prongs spatially distributed in accordance with a target artificial vasculature. When positioned onto the pronged component, the receiver component defines the 3D-cell culture cavity of the mold while the pronged component lets plurality of prongs into the 3D-cell culture cavity. The mold is exposed to solidification conditions of the liquid (e.g., hydrogel) for obtained the 3D-cell culture embedding the target artificial vasculature.

Optionally, in addition or alternatively the method may comprise allowing the pronged component to protrude through the 3D-cell culture cavity by positioning the receiver component onto the pronged component.

Optionally, in addition or alternatively the method may comprise pouring the polymerizable liquid (e.g., hydrogel) into the 3D-cell culture cavity through an opening and stanching the 3D-cell culture cavity to the polymerizable liquid by positioning the receiver component over the pronged component.

Optionally, in addition or alternatively the method may comprise filling the 3D-cell culture cavity with a volume between 0.5 cubic centimeter and 1.5 cubic centimeters of the polymerizable liquid (e.g., hydrogel).

Optionally, in addition or alternatively, the method may comprise pouring the polymerizable liquid through an additional hole in the base of the receiver component.

Optionally, in addition or alternatively, the method may comprise separating the pronged component and the receiver component once the liquid has solidified by using a holder portion of the mold.

In a third aspect, the techniques described herein relate to a system for preparing a 3D-cell culture with improved nutrient diffusion, oxygen supply and/or waste removal from cells embedded therein. The system comprises a mold, a pouring module and an exposition module. The mold comprises a pronged component and a receiver component. The pronged component of the mold comprises a plurality of prongs. The plurality of prongs is spatially distributed in accordance with a target artificial vasculature. The receiver component of the mold defines a 3D-cell culture cavity thereof when positioned onto the pronged component. The pronged component lets the plurality of prongs into the 3D-cell culture cavity. The pouring module is configured to controllably pour a polymerizable liquid (e.g., hydrogel) into the mold. The exposition module is configured to expose the mold to solidification conditions of the liquid (e.g., hydrogel) thereby obtaining the 3D-cell culture embedding the target artificial vasculature.

Optionally, in addition or alternatively, the plurality of prongs protrudes through the 3D-cell culture cavity when the receiver component is positioned onto the pronged component of the mold. Optionally, in addition or alternatively, an opening in the mold is defined to receive the liquid (e.g., hydrogel) towards the 3D-cell culture cavity and the 3D cell culture cavity is stanched to the liquid when the receiver component is operably positioned over the pronged component. Optionally, in addition or alternatively, the 3D-cell culture cavity defines a volume to receive between about 0.5 cubic centimeter to about 1.5 cubic centimeters of the liquid. Optionally, in addition or alternatively, each of the plurality of prongs protrudes through the receiver component through an individual hole in the receiver component and, in such a scenario, the polymerizable liquid (e.g., hydrogel) may or may not be poured into the mold using an additional hole in a base of the receiver component towards the 3D-cell culture cavity. Optionally, in addition or alternatively, the pronged component and the receiver component are separated after solidification of the liquid using a holder.

In various aspects and embodiments, the present disclosure also provides the following items 1 to 26:
1. A 3D-cell-culture preparation device comprising:
   a pronged component comprising a plurality of outwards prongs, the plurality of prongs being spatially distributed in accordance with a target artificial vasculature; and
   a receiver component defining a 3D-cell culture cavity when positioned onto the pronged component, the pronged component letting the plurality of prongs into the 3D-cell-culture cavity,
   wherein the device is configured to receive a polymerizable liquid in the 3D-cell culture cavity.
2. The device of item 1, wherein the plurality of prongs protrudes through the 3D-cell culture cavity once the receiver component is positioned onto the pronged component.
3. The device of item 1 or 2, wherein an opening is defined to receive the liquid toward the 3D-cell culture cavity and the 3D cell culture cavity is stanched to the liquid when the receiver component is operably positioned over the pronged component.
4. The device of any one of items 1 to 3, wherein the 3D-cell culture cavity defines a volume to receive between 0.5 cubic centimeter and 1.5 cubic centimeters of the liquid.
5. The device of any one of items 1 to 4, wherein the receiver component comprises a hole in a base thereof for each of the plurality of prongs.
6. The device of item 5, wherein at least one additional hole in the base of the receiver component is left unoccupied by the plurality of prongs.
7. The device of item 6, wherein the at least one additional hole in the base of the receiver component is located at one or more of the corners of the base of the receiver component.
8. The device of any one of items 1 to 7, further comprising a holder portion configured to facilitate separation of the receiver component and pronged component.
9. The device of any one of items 1 to 8, wherein:
   the spatial distribution leaves at least 0.5 mm between each of the plurality of prongs; and
   the plurality of prongs occupies between 10% and 25% of a culture volume of the 3D-cell culture cavity, the culture volume being defined by a volume of liquid received in the 3D-cell culture cavity.
10. The device of any one of items 1 to 9, wherein the plurality of prongs are shaped as one or more of a cylinder, a polygonal base prism, an elliptical prism, a histologically-inspired prism and an enamel-inspired prism.
11. A method for preparing a 3D cell culture, the method comprising:
   (a) pouring a polymerizable liquid into a mold, the mold comprising:
      (i) a pronged component comprising a plurality of prongs, the plurality of prongs being spatially distributed in accordance with a target artificial vasculature; and
      (ii) a receiver component defining a 3D-cell culture cavity of the mold when positioned onto the pronged component, the pronged component letting the plurality of prongs into the 3D-cell culture cavity;
   (b) exposing the mold to solidification conditions of the polymerizable liquid thereby obtaining the 3D-cell culture embedding the target artificial vasculature.
12. The method of item 11, further comprising positioning the receiver component onto the pronged component, wherein the plurality of prongs protrudes through the 3D-cell culture cavity.
13. The method of item 11 or item 12, further comprising pouring the polymerizable liquid into the 3D cell culture cavity through an opening and stanching the 3D-cell culture cavity to the polymerizable liquid by positioning the receiver component over the pronged component.
14. The method of any one of items 11 to 13, wherein a culture volume of the 3D-cell culture cavity is between 0.5 cubic centimeter and 1.5 cubic centimeters of the polymerizable liquid.
15. The method of any one of items 11 to 14, wherein each of the plurality of prongs protrude through the receiver component across an individual hole.
16. The method of item 15, further comprising pouring the liquid through an additional hole in a base of the receiver component towards the 3D-cell culture cavity.
17. The method of any one of items 11 to 16, further comprising separating the pronged component and the receiver component once the polymerizable liquid has solidified using a holder portion of the mold.
18. The method of any one of items 11 to 17, wherein the plurality of prongs:
   (a) is distributed at a distance of at least 0.1 or 0.2 mm between each other; and
   (b) occupies between 10% and 25% of a culture volume of the 3D-cell culture cavity, the culture volume being defined by a volume of liquid received in the 3D-cell culture cavity.
19. The method of any one of items 11 to 18, wherein each of the plurality of prongs is shaped as one or more of a cylinder, a polygonal base prism, an elliptical prism, a histologically-inspired prism and an enamel-inspired prism.
20. A system for preparing a 3D-cell culture, the system comprising:
   (a) a mold comprising:
      (i) a pronged component comprising a plurality of prongs, the plurality of prongs being spatially distributed in accordance with a target artificial vasculature; and
      (ii) a receiver component defining a 3D-cell culture cavity of the mold when positioned onto the pronged component, the pronged component letting the plurality of prongs into the 3D-cell culture cavity;
   (b) a pouring module configured to controllably pour a polymerizable liquid into the mold;
   (c) an exposition module configured to expose the mold to solidification conditions of the liquid thereby obtaining the 3D-cell culture embedding the target artificial vasculature.
21. The system of item 20, wherein the plurality of prongs protrudes through the 3D-cell culture cavity when the receiver component is positioned onto the pronged component of the mold.
22. The system of item 20 or 21, wherein an opening in the mold is defined to receive the liquid toward the 3D-cell culture cavity and the 3D-cell culture cavity is stanched to the liquid when the receiver component is operably positioned over the pronged component.
23. The system of any one of items 20 to 22, wherein the 3D-cell culture cavity defines a volume to receive between 0.5 cubic centimeter and 1.5 cubic centimeters of the liquid.
24. The system of any one of items 20 to 23, wherein each of the plurality of prongs protrude through the receiver component through an individual hole in the receiver component.
25. The system of item 24, wherein the polymerizable liquid is poured into the mold using an addition hole in a base of the receiver component towards the 3D-cell culture cavity.
26. The system of any one of items 20 to 25, wherein the pronged component and the receiver component are separated after solidification of the liquid using a holder.

Other objects, advantages and features of the present disclosure will become more apparent upon reading of the following non-restrictive description of specific embodiments thereof, given by way of example only with reference to the accompanying drawings.

### BRIEF DESCRIPTION OF DRAWINGS

Further features and exemplary advantages of the present disclosure will become apparent from the following detailed description, taken in conjunction with the appended drawings, in which:

FIGS. 1 to 4 are views of exemplary devices in accordance with the teachings of the present disclosure;

FIG. 5 is a flowchart of an exemplary method in accordance with the teachings of the present disclosure;

FIG. 6 is a logical modular representation of an exemplary network node deployed in a system in accordance with the teachings of the present disclosure;

FIGS. 7A and 7B are images of stained A549 cells (lung cancer, FIG. 7A) and MDA-MB-231 cells (breast cancer, FIG. 7B) in cryosections of the 3D gel prepared using the device and method in accordance with the teachings of the present disclosure. Cells were stained with DAPI (nucleus), Ki67 (marker of proliferation), and HIF1α (marker of hypoxia) after 1 week, 2 weeks or 4 weeks in culture. Leica inverted microscope, objective 40×;

FIG. 8 depicts images showing the permeability of the 3D gel prepared using the device and method in accordance with the teachings of the present disclosure;

FIG. 9A is a schematic of the assay used to analyze the diffusion of a cell-permeable fluorescent dye in the 3D gel prepared using the device and method in accordance with the teachings of the present disclosure;

FIG. 9B is a microscopic image of cells in the capillary stained by the cell-permeable fluorescent dye (1 h incubation, objective 20×);

FIG. 9C is a graph showing the flow cytometry data obtained on stained human cancer cells by the cell-permeable fluorescent dye (CFSE positive cells) after 1 h, 2 h and 4 h. The percentage of CFSE positive cells (CSFE+) is shown in cells obtained from the internal or external sections of the 3D gel prepared using the device and method in accordance with the teachings of the present disclosure.

### DETAILED DISCLOSURE

The use of the terms “a” and “an” and “the” and similar referents in the context of describing the technology (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

All methods described herein can be performed in any suitable order unless otherwise indicated herein or otherwise clearly contradicted by context.

The use of any and all examples, or exemplary language (“e.g.”, “such as”) provided herein, is intended merely to better illustrate embodiments of the claimed technology and does not pose a limitation on the scope unless otherwise claimed.

No language in the specification should be construed as indicating any non-claimed element as essential to the practice of embodiments of the claimed technology.

Herein, the term “about” has its ordinary meaning. The term “about” is used to indicate that a value includes an inherent variation of error for the device or the method being employed to determine the value, or encompass values close to the recited values, for example within 10% of the recited values (or range of values).

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Where features or aspects of the disclosure are described in terms of Markush groups or list of alternatives, those skilled in the art will recognize that the disclosure is also thereby described in terms of any individual member, or subgroup of members, of the Markush group or list of alternatives.

Unless specifically defined otherwise, all technical and scientific terms used herein shall be taken to have the same meaning as commonly understood by one of ordinary skill in the art (e.g., in stem cell biology, cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

Most cell culture is performed in two-dimensional (2D) petri dishes or tissue culture flasks. In these conditions, cells are grown, in monoculture, at low density and exposed to an artificial microenvironment, which is detrimental to cell behavior, phenotype and drug responses. Unrealistic 2D conditions translate into the poor performance of preclinical models to study biology and identify drugs that will be effective in patients. As an example, 90% of all drugs tested in clinical trials will never be approved due to lack of efficacy. In oncology, the success rate is lower with only 5% of all tested anticancer drugs in clinical trials, being granted with approval. In the case of Alzheimer disease, the success rate is less than 1%. This is an enormous waste of resources; time and it is ethically questionable to enroll patients in clinical trials with such a low success rate.

Thus, there is a high need to provide cell culture tools that are more representative of human tissues for basic and translational studies. 2D culture lacks the three-dimensional (3D) architecture that affects cell proliferation, metabolism, gene expression and the epigenome. Thereby, replicating a relevant microenvironment may re-establish a relevant cell phenotype, through cell plasticity mechanisms—including effects on the epigenome.

As such, interest in 3D cell culture is increasing. Most 3D tissue culture systems rely on expensive 3D bio-printers and organ-on-chip technologies that are limited to specific cell types and restricted to a limited group of experts. Other 3D culture systems are based on the development of spheroid or organoid culture, which are often limited in size, growth potential and are technically challenging. There is a need for innovation to bring affordable and accessible 3D culture to grow tissue-like structures in vitro.

A 3D cavity to perform 3D culture supporting a target artificial vasculature is provided herein. Example of target advantages of the technology is affordability, ease of use and suitability for more than one cell type. The cavity with vasculature (or scaffold) approach has been demonstrated to grow tissue-like structure of 1 cubic centimeter presenting pseudo-capillaries in accordance with the target vasculature that enhances oxygen and nutrient diffusion within the in vitro 3D tissue.

While some embodiments presented herein may not answer to all aspects of an optimized solution, initial requirements for the technology included a volume at least about 1 cubic centimeter, structure built with sections separated by artificial blood vessels (e.g., accessible to fill endothelial cells that will grow along the artificial blood vessels), multiple injection sites allowing the filling of cell mixture (cancer cells+endothelial cells (e.g., fibroblasts)+nerve cells+immune cells), use with hydrogel materials with biomechanical properties mimicking tissue that are trypsin or collagenase sensitive allowing enzymatic digestion for downstream experiments, connectivity with one or more additional structure (e.g., of about 1 cubic centimeter) for different purposes such as mimicking metastasis.

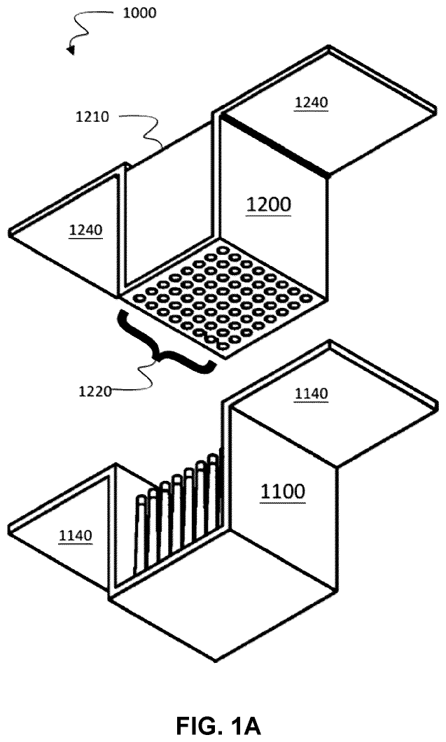

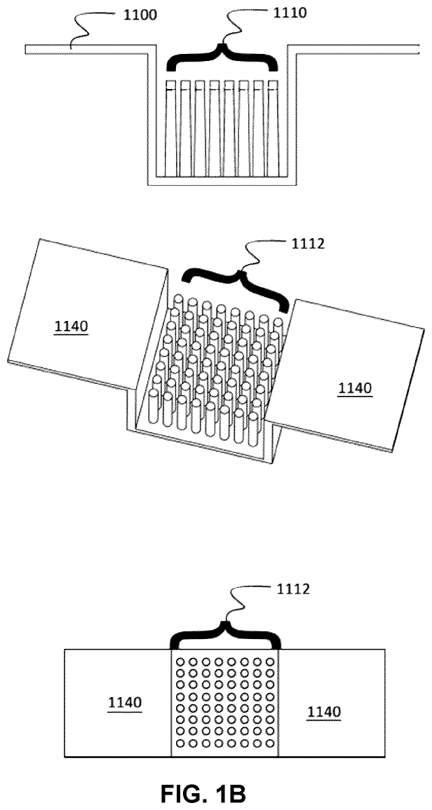

Reference is now made to the drawings concurrently to FIGS. 1-4, which show examples of a 3D cell culture preparation device 1000. The device 1000 comprises a prong component 1100 and a receiver component 1200. The pronged component 1100 comprises a plurality of prongs 1110. The plurality of prongs 1110 is spatially distributed in accordance with the target artificial vasculature. The receiver component 1200 defines a 3D cell culture cavity 1210 when positioned onto the pronged component 1100. The plurality of prongs 1110 are thereby positioned into the 3D cell culture cavity 1210. The device 1000 is configured to receive a liquid in the 3D cell culture cavity 1210.

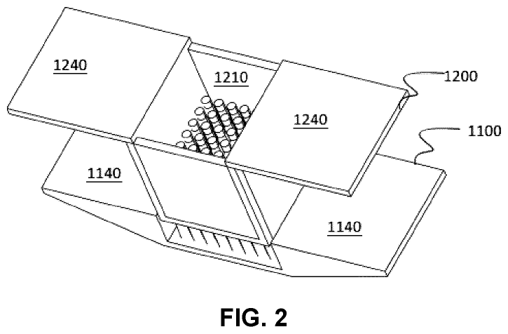

In some embodiments, as depicted on FIG. 2, the plurality of prongs 1110 protrude through the 3D cell culture cavity 1210 once the receiver component 1200 is positioned onto the pronged component 1100. As depicted in the example of FIG. 2, an opening is defined on one surface of the 3D cell culture cavity 1210 to receive the liquid theretoward. In the example, the 3D cell culture cavity 1210 is stanched to the liquid when the receiver component 1200 is operably positioned over the pronged component 1100. The example of FIG. 2 also provides other examples of shapes of the pronged component 1100 in relation to the holder portions 1240 and/or 1140.

The volume of the 3D cell culture cavity may be defined to receive between about 0.5 cubic centimeter to about 1.5 cubic centimeters of the liquid. The receiver component 1200 may comprise a hole 1220 in a base thereof for each of the plurality of prongs 1110. In such a case, optionally, at least one additional hole (not shown) in the base of the receiver component 1200 may further be left unoccupied by the plurality of prongs 1110 such as to allow the liquid to be injected or inserted into the 3D cell culture cavity 1210.

The 3D cell culture preparation device 1000 may further comprise a holder portion (e.g., 1240 and/or 1140) configured to facilitate separation of the receiver component 1200 and pronged component 1100.

The size/diameter of the prongs and/or the spaces between the prongs may be adapted to mimic the vasculature of the target organ or tissue (i.e., the organ or tissue that is being reproduced/mimicked by the 3D cell culture). The size/diameter of the prongs and/or the spaces between the prongs should be adapted to provide a 3D cell structure that is sufficiently rigid to maintain its integrity once removed from the device and put into culture, but at the same time that provide sufficient oxygen and nutrient diffusion throughout the 3D cell structure. In an embodiment, the spatial distribution leaves at least about 0.1 or 0.2 mm, for example about 0.4 mm, between each of the plurality of prongs 1110. The plurality of prongs 1100 may occupy between about 5 to about 50%, about 5 to about 40%, about 10 to about 30% or about 10% to about 25% of a culture volume of the 3D-cell-culture cavity 1210. The culture volume is defined by a volume of liquid received in the 3D cell culture cavity 1210. Optionally, in addition or alternatively, the spatial distribution leaves about 0.4 mm to about 0.8 mm between each of the plurality of prongs, for example about 0.5 mm to about 0.7 mm, about 0.55 mm to about 0.65 mm, about 0.56 mm to about 0.64 mm, about 0.57 mm to about 0.63 mm, about 0.58 mm to about 0.62 mm, about 0.59 mm to about 0.61 mm, or about 0.6 mm. Optionally, in addition or alternatively, the size/diameter of the prongs is from about 0.5 mm to about 1.5 mm, from about 0.6 mm to about 1.4 mm, from about 0.7 mm to about 1.3 mm, from about 0.8 mm to about 1.2 mm, from about 0.9 mm to about 1.1 mm, or about 1 mm. It is to be understood that the prongs may be identical (same size/diameter) or may have different sizes/diameters. Similarly, the spaces between the prongs may be the same or different.

The plurality of prongs 1110 may be shaped as a cylinder 1112 and/or truncated cone 1110 as depicted on FIGS. 1, 2 and 4. Alternatively or additionally plurality of prongs 1110 may be shaped as a polygonal base prism, an elliptical prism, a histologically-inspired prism and an enamel-inspired prism. Examples are provided in relation to the pronged component 1100′ on FIG. 4, which may be readily modified in the context of the examples of FIG. 1 and FIG. 2. The term histologically-inspired prism as used herein refers to a geometric shape that has features or an organization that mimic or are influenced by the microscopic structures of tissues, and more particularly blood vessels such as veins and capillaries.

In an embodiment, the height of the prongs is from about 8 to about 16 mm, for example from about 10 and about 14 mm, from about 11 to about 13 mm, or about 12 mm. The height of the different prongs may be the same or different, preferably the height of the different prongs is the same.

Initial tests were performed with the liquid being different hydrogels (alginate and Gel-MA) with the different prototypes. The tests allowed the determination of preferred heights and preferred number of prongs in the pronged component 1110. The liquid may be selected, as in the experiments described herein, for polymerization and extraction characteristics.

In an embodiment, the liquid is a biocompatible hydrogel or a mixture of different hydrogels. “Biocompatible” as used herein means that the hydrogel is not toxic for the cells. The hydrogel comprises water and a hydrophilic polymer. The hydrophilic polymer is present in a concentration such that a gel is obtained, which concentration will depend on the exact nature of the polymer used as well as the desired mechanical properties of the hydrogel (e.g., strength, viscosity). Non-limiting examples of hydrophilic polymers for hydrogel include collagen, fibrin, fibronectin, hyaluronic acid, gelatin, alginate, carboxymethylcellulose (CMC), guar gum, gellan gum, agarose and the gelatinous protein mixture secreted by Engelbreth-Holm-Swarm (EHS) mouse sarcoma cells (commercially available from Corning Life Sciences, Millipore Sigma and BD Biosciences as Matrigel® and from Trevigen, Inc. Cultrex® BME), de-cellularized patient extracellular matrix (which contains extracellular matrix components such as collagen, fibronectin, laminin, glycosaminoglycans, and other biological molecules including growth factors), PEG, hydroxyapatite, chitosan, as well as mixtures thereof. The hydrogel may also comprise polymers and copolymers with an abundance of hydrophilic group such as polyvinyl alcohol polymers and copolymers, sodium polyacrylate polymers and copolymers, acrylate polymers and copolymers, or any combination thereof. In an embodiment, the hydrogel is sensitive to chemical or enzymatic digestion, e.g., by trypsin or collagenase.

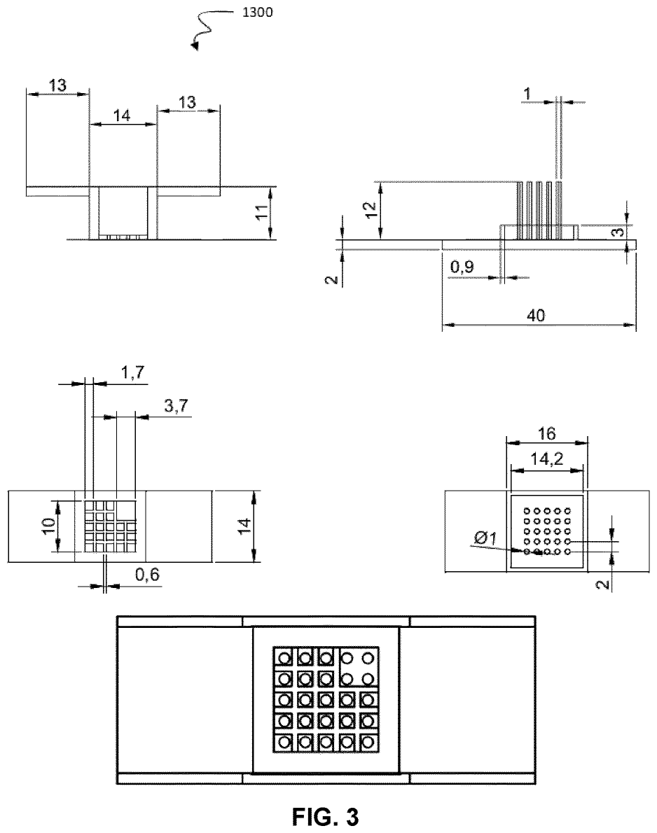

One exemplary device 1300 that was found satisfactory is depicted on FIG. 3 and comprises wider wings 1540 to allow easier manipulation. The pronged component 1400 may contain 15 prongs 1410 and measure 16×40 mm. The receiver component 1500 may contain a grid 1520 with a hole 1530 in the grid section, for example in one of the corners (to facilitate the detachment of the hydrogel from the component), and measure 14×40 mm.

One exemplary device 1400 on FIG. 4 presents other examples of shaped for the prongs such as one or more of a cylinder, a polygonal base prism, an elliptical prism, a histologically-inspired prism and an enamel-inspired prism. Furthermore, the device 1400 depicted on FIG. 4 provides a pronged component 1100′ positioned under a receiver component 1200′. The 3D cell culture cavity may then be filled using one or more holes 1410 in the receiver component 1200′. In the depicted example, the prongs are inserted into depression in the receiver component 1200′ That is, the device 1400 depicts an example where the prongs are inserted into the 3D-cell-culture cavity without protruding through the receiver component 1200′. Furthermore, as exemplified, the material of the device 1400 may completely or partially be transparent or translucid, which may be useful depending on the solidification conditions of the polymerizable liquid. In cases where the receiver component 1200′ is below the pronged component upon filling, a cover 1420 may be used to avoid a leak through the hole 1410 (e.g., obstructing the hole 1410 using a corresponding peg 1422 of the cover 1420).

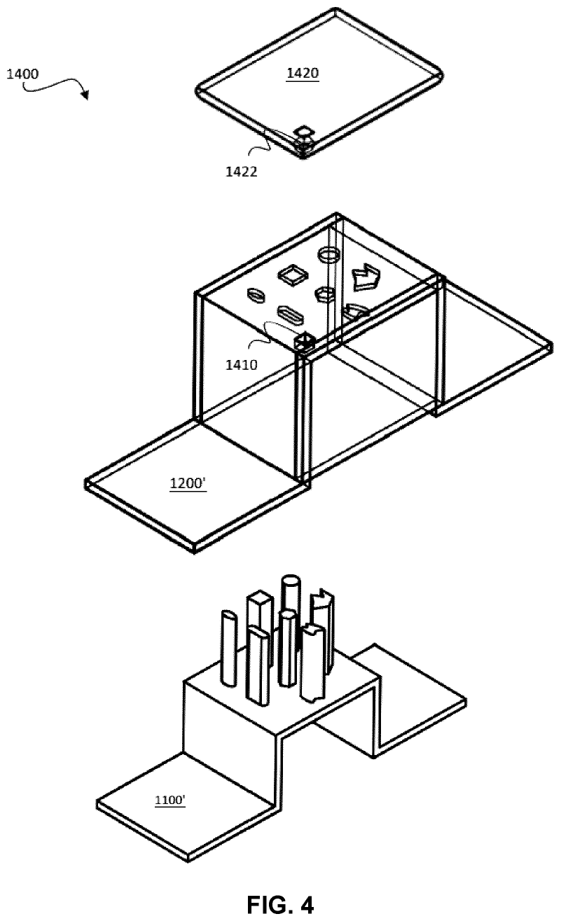

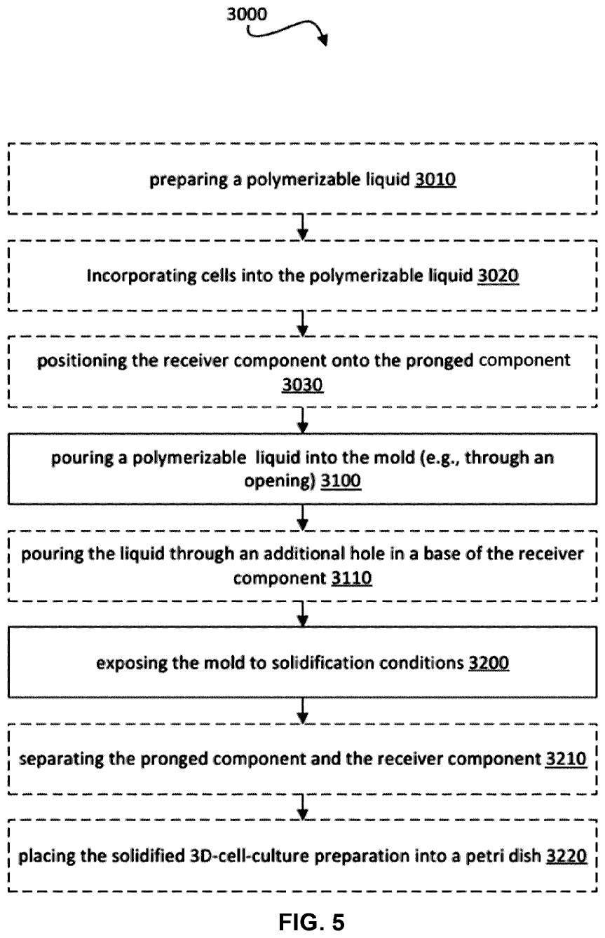

A method 3000 is depicted on FIG. 5 for preparing a 3D cell culture with improved nutrient diffusion, oxygen supply and/or waste removal from cells embedded therein. A polymerizable liquid is poured 3100 into a mold corresponding to the device 1000. That is, the mold comprises a pronged component and a receiver component. The pronged component comprises a plurality of prongs spatially distributed in accordance with a target artificial vasculature. When positioned onto the pronged component, the receiver component defines the 3D cell culture cavity of the mold while the pronged component lets plurality of prongs into the 3D cell culture cavity. The mold is exposed 3200 to solidification conditions of the liquid for obtained the 3D cell culture embedding the target artificial vasculature. Solidification conditions may include temperature (e.g., low temperature), light (e.g., UV light), pH (change of pH inducing polymerization), chemicals (e.g., cross-linking agent), or any combinations thereof.

Optionally, stanching the 3D cell culture cavity to the polymerizable liquid may be performed by positioning 3030 the receiver component over the pronged component. Pouring 3100 the polymerizable liquid into the 3D cell culture cavity may be performed through an opening of the 3D-cell-culture cavity. The 3D cell culture cavity may provide a volume between 0.5 cubic centimeter and 1.5 cubic centimeters of the polymerizable liquid. Optionally, in addition or alternatively, the method 3000 may comprise pouring 3110 the polymerizable liquid through an additional hole in the base of the receiver component. The method may further comprise separating 3210 the pronged component and the receiver component once the liquid has solidified by using a holder portion of the mold.

In some embodiments, the method 3000 may further comprise preparing 3010 the polymerizable liquid. The preparing may be performed, for instance, by reconstituting 500 mg Methacrylated Gelatin (Advanced Biomatrix) in 5 mL appropriated media supplemented with 10% FBS. As skilled persons will readily recognize, the media composition depends on the cell line to be cultured. The preparing 3010 may also comprise warming the hydrogel in 37° C. bath until complete dissolution. Skilled persons will recognize that the warming may can take several hours. 0.5% Lithium phenyl-2,4,6-trimethylbenzoylphosphinate (LAP) may be added (e.g., just before pouring 3100) before mixing up and down and waiting to obtain a homogeneous solution. Once the polymerizable liquid is a homogeneous solution, it may be kept in a warm and dark place.

Depending on solidification conditions of the polymerizable liquid, it may be best preparing 3010 only the necessary volume and performing the preparing 3010 in a dark environment (e.g., not exposing the liquid to solidification conditions).

The method 3000 may further comprise incorporating cells into the polymerizable liquid. The incorporating 3020 may comprise washing the cells with 1×PBS and add 0.25% trypsin to detach them from the petri dish before incubating the cells at 37° C. The incorporating may also comprise inactivating the trypsin with media supplemented with 10% FBS. It is then possible to count cells and pellet by centrifugation the correct amount of cells before discarding the supernatant. As skilled persons will readily recognize, different cell lines may be used in co-culture. Likewise, the number of cells can be adjusted as needed according to the application. The incorporating 3020 may comprise mixing gently up and down, 5 million cells per 1 ml polymerizable liquid (e.g., being careful not to make bubbles).

Any type of cells (or combination of cells) suitable to mimic a tissue or organ of interest may be used. Thus, the type(s) of cells is selected according to the tissue or organ that the cell culture system intends to mimic. The cells may be, e.g., connective tissue cells (e.g., stromal cells, fibroblasts), endothelial cells, epithelial cells, neuroglial cells, neurons, muscle cells (e.g., skeletal, cardiac, or smooth muscle cells), cartilage cells (e.g., chondrocytes), bone cells (e.g., osteoblasts, osteoclasts, osteocytes, lining cells), skin cells (e.g., keratinocytes, melanocytes, Langerhans cells), immune cells (e.g., lymphocytes, macrophages/monocytes, neutrophils, etc.), astrocytes, induced-pluripotent stem cells, stem cells, cancer stem cells, or any combination thereof, the list of course not being exhaustive. The cells may be primary cells, a cell line, malignant or non-malignant (normal) cells, genetically-engineered cells, etc. In an embodiment, the cells comprise tumor or malignant cells, primary tumor cells or a tumor cell line. In an embodiment, the tumor cells are non-metastatic, i.e., have no metastasis potential. In another embodiment, the tumor cells are metastatic. The tumor cell may be a heart sarcoma cell, lung cancer cell, small cell lung cancer (SCLC) cell, non-small cell lung cancer (NSCLC) cell, bronchogenic carcinoma cell (squamous cell, undifferentiated small cell, undifferentiated large cell, adenocarcinoma), alveolar (bronchiolar) carcinoma cell, bronchial adenoma cell, sarcoma cell (e.g., Ewing's sarcoma, Karposi's sarcoma), chondromatous hamartoma cell, mesothelioma cell; cancer cell of the gastrointestinal system, for example, esophagus cancer cell (squamous cell carcinoma, adenocarcinoma, leiomyosarcoma, lymphoma), stomach cancer cell (carcinoma, lymphoma, leiomyosarcoma), gastric cancer cell, pancreas cancer cell (ductal adenocarcinoma, insulinoma, glucagonoma, gastrinoma, carcinoid tumors, vipoma), small bowel cancer cell (adenocarcinoma, lymphoma, carcinoid tumors, Karposi's sarcoma, leiomyoma, hemangioma, lipoma, neurofibroma, fibroma), large bowel cancer cell (adenocarcinoma, tubular adenoma, villous adenoma, hamartoma, leiomyoma); cancer cell of the genitourinary tract, for example, kidney cancer cell (adenocarcinoma, Wilm's tumor cell [nephroblastoma], lymphoma), bladder cancer cell and/or urethra cancer cell (squamous cell carcinoma, transitional cell carcinoma, adenocarcinoma), prostate cancer cell (adenocarcinoma, sarcoma), testis cancer cell (seminoma, teratoma, embryonal carcinoma, teratocarcinoma, choriocarcinoma, sarcoma, interstitial cell carcinoma, fibroma, fibroadenoma, adenomatoid tumors, lipoma); liver cancer cell, for example, hepatoma (hepatocellular carcinoma, HCC), cholangiocarcinoma, hepatoblastoma, angiosarcoma, hepatocellular adenoma, hemangioma, pancreatic endocrine tumors (such as pheochromocytoma, insulinoma, vasoactive intestinal peptide tumor, islet cell tumor and glucagonoma); bone cancer cell, for example, osteogenic sarcoma (osteosarcoma), fibrosarcoma, malignant fibrous histiocytoma, chondrosarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; cancer cell of the nervous system, for example, neoplasms of the central nervous system (CNS), primary CNS lymphoma, skull cancer (osteoma, hemangioma, granuloma, xanthoma, osteitis deformans), meninges (meningioma, meningiosarcoma, gliomatosis), brain cancer cell (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); cancer cell of the reproductive system, for example, gynecological cancer cell, uterine cancer cell (endometrial carcinoma), cervical cancer cell (cervical carcinoma, pre-tumor cervical dysplasia), ovarian cancer cell (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulvar cancer cell (squamous cell carcinoma, intraepithelial carcinoma, adenocarcinoma, fibrosarcoma, melanoma), vaginal cancer cell (clear cell carcinoma, squamous cell carcinoma, botryoid sarcoma (embryonal rhabdomyosarcoma), fallopian tube cancer cell (carcinoma); placenta cancer cell, penile cancer cell, prostate cancer cell, testicular cancer cell; cancer cell of the oral cavity, for example, lip cancer cell, tongue cancer cell, gum cancer cell, palate cancer cell, oropharynx cancer cell, nasopharynx cancer cell, sinus cancer cell; skin cancer cell, for example, malignant melanoma, cutaneous melanoma, basal cell carcinoma, squamous cell carcinoma, Karposi's sarcoma, moles dysplastic nevi, lipoma, angioma, dermatofibroma, and keloids; adrenal gland cancer cell: neuroblastoma; and cancer cells of other tissues including connective and soft tissue tumors, retroperitoneum and peritoneum, eye cancer cell, intraocular melanoma, and adnexa, breast cancer cell (e.g., ductal breast cancer), head or/and neck cancer cell (head and neck squamous cell carcinoma), anal cancer cell, thyroid cancer cell, cancer stem cell, and parathyroid cancer cell. In another embodiment, the cells comprise at least one type of cancer cells and at least one type of non-cancer cells. In an embodiment, the non-cancer cells are cells found in a tumor, i.e., cells present in a tumor but that are not the malignant cancer cells per se (sometimes referred to as tumor-associated cells), including cancer-associated fibroblasts (CAFs), tumor-associated immune cells such as tumor-associated macrophages (TAMs) and tumor-infiltrating lymphocytes (TIFs).

The pouring 3100 may then be performed by filling the mold with 1 mL of the cell/hydrogel suspension prepared in 3020 (or with different combination of cell/hydrogel suspensions) before exposing 3200 one or more filled the molds to 405 nm light during 1-1.5 min or until the polymerizable liquid whitens. It will be recognized that the time is given as an indication and must be adjusted according to the light intensity and the distance between the mold and the light source. In experiments performed, polymerization in 1 min was obtained with a 405 nm LED UV light 60W placed 10 cm above the molds.

The mold may then be separated 3210 (e.g., using the holder portions). Depending on the structure of the mold, the solidified 3D-cell-culture preparation may remain in the upper part (or receiver component). The method 3000 may then continue with placing 3220 the solidified 3D cell culture preparation into a petri dish. The placing 3220 may be performed by inverting the receiver component over a petri dish and inserting a tool (e.g., cotton swab, pipet tip) into a hole provided for this purpose therein, preferably a hole located in one of the corners of the received component. The solidified 3D cell culture preparation is thereby pushed into the petri dish. Different 3D-cell-culture preparations (e.g., comprising different cells and/or hydrogels) may be put into the petri dish.

Additional media may then be added (e.g., to achieve a target height) before placing petri dish in an incubator (37° C., 5% CO₂ for standard conditions or in hypoxic chambers or other defined conditions for cell incubation).

In exemplary experiments, using the device 1000 (a.k.a. Alveocell scaffold), it was possible to culture, in a hydrogel mimicking tissue collagen, several cancer cell lines (from breast, lung tumors) alone or with a combination of supportive cells such as fibroblasts (MRC5) and endothelial cells (HUVEC). Cell viability within the device 1000 ranged from 60 to 70% measured at 1 week and after 4 weeks in culture. It is expected that the device 1000 may be used for co-culture with human cancer cells, fibroblasts, endothelial cells, and/or immune cells.

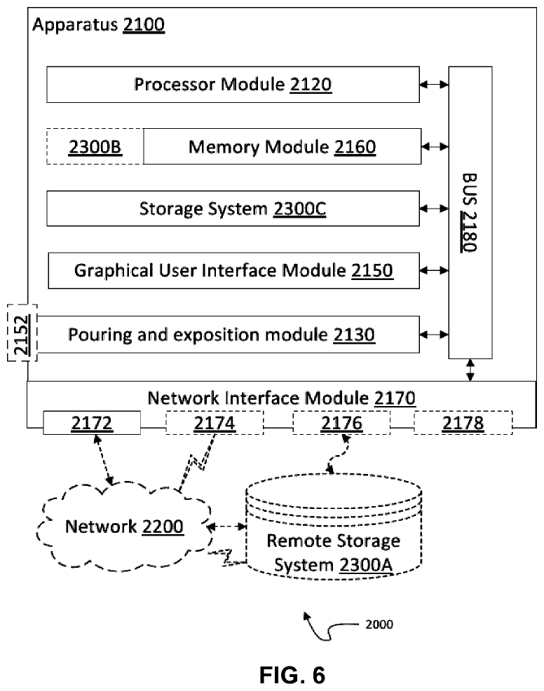

Reference is now made to the drawings in which FIG. 6 shows a logical modular representation of an exemplary system 2000 comprising an apparatus 2100. The apparatus 2100 comprises a memory module 2160, a processor module 2120, a pouring and exposition module 2130 and a network interface module 2170. The apparatus 2100 may also include a user interface module 2150.

The system 2000 is configured for preparing a 3D cell culture with improved nutrient diffusion, oxygen supply and/or waste removal from cells embedded therein. The system 2000 comprises a mold similar to the device 1000. The mold comprises a pronged component and a receiver component. The pronged component of the mold comprises a plurality of prongs. The plurality of prongs is spatially distributed in accordance with a target artificial vasculature. The receiver component of the mold defines a 3D cell culture cavity thereof when positioned onto the pronged component. The pronged component lets the plurality of prongs into the 3D cell culture cavity. The pouring and exposition 2130 module is configured to controllably pour a polymerizable liquid into the mold. The pouring and exposition 2130 is also configured to expose the mold to solidification conditions of the liquid thereby obtaining the 3D cell culture embedding the target artificial vasculature.

The system 2000 may comprise a storage system 2300 for storing and accessing long-term (i.e., non-transitory) data and may further log data while the apparatus 2100 is being used. FIG. 6 shows examples of the storage system 2300 as a distinct database system 2300A, a distinct module 2300C of the apparatus 2100 or a sub-module 2300B of the memory module 2160 of the apparatus 2100. The storage system 2300 may be distributed over different systems A, B, C. The storage system 2300 may comprise one or more logical or physical as well as local or remote hard disk drive (HDD) (or an array thereof). The storage system 2300 may further comprise a local or remote database made accessible to the apparatus 2100 by a standardized or proprietary interface or via the network interface module 2170.

The network interface module 2170 represents at least one physical interface that can be used to communicate with other network nodes. The network interface module 2170 may be made visible to the other modules of the apparatus 2100 through one or more logical interfaces. The actual stacks of protocols used by the physical network interface(s) and/or logical network interface(s) 2172-2178 of the network interface module 2170 do not affect the teachings of the present disclosure.

The processor module 2120 may represent a single processor with one or more processor cores or an array of processors, each comprising one or more processor cores. The memory module 2160 may comprise various types of memory (different standardized or kinds of Random Access Memory (RAM) modules, memory cards, Read-Only Memory (ROM) modules, programmable ROM, etc.).

A bus 2180 is depicted as an example of means for exchanging data between the different modules of the apparatus 2100. The teachings presented herein are not affected by the way the different modules exchange information. For instance, the memory module 2160 and the processor module 2120 could be connected by a parallel bus, but could also be connected by a serial connection or involve an intermediate module (not shown) without affecting the teachings of the present disclosure.

The pouring and exposition module 2130 provides pouring and exposition-related services to the apparatus 2100 (i.e., see the method 3000).

The variants of processor module 2120, memory module 2160 and network interface module 2170 usable in the context of the present disclosure will be readily apparent to persons skilled in the art. Likewise, even though explicit mentions of the pouring and exposition module 2130, the memory module 2160, the user interface module 2150 and/or the processor module 2120 are not made throughout the description of the present examples, persons skilled in the art will readily recognize when such modules are used in conjunction with other modules of the apparatus 2100 to perform routine as well as innovative elements presented herein.

Various network links may be implicitly or explicitly used in the context of the present disclosure. While a link may be depicted as a wireless link, it could also be embodied as a wired link using a coaxial cable, an optical fiber, a category 5 cable, and the like. A wired or wireless access point (not shown) may be present on the link between. Likewise, any number of routers (not shown) may be present and part of the link, which may further pass through the Internet.

The present invention is not affected by the way the different modules exchange information between them. For instance, the memory module and the processor module could be connected by a parallel bus, but could also be connected by a serial connection or involve an intermediate module (not shown) without affecting the teachings of the present disclosure.

A method is generally conceived to be a self-consistent sequence of steps leading to a desired result. These steps require physical manipulations of physical quantities. Usually, though not necessarily, these quantities take the form of electrical or magnetic/electromagnetic signals capable of being stored, transferred, combined, compared, and otherwise manipulated. It is convenient at times, principally for reasons of common usage, to refer to these signals as bits, values, parameters, items, elements, objects, symbols, characters, terms, numbers, or the like. It should be noted, however, that all of these terms and similar terms are to be associated with the appropriate physical quantities and are merely convenient labels applied to these quantities. The description of the present invention has been presented for purposes of illustration but is not intended to be exhaustive or limited to the disclosed embodiments. Many modifications and variations will be apparent to those of ordinary skill in the art. The embodiments were chosen to explain the principles of the invention and its practical applications and to enable others of ordinary skill in the art to understand the invention in order to implement various embodiments with various modifications as might be suited to other contemplated uses.

### EXAMPLES

The present disclosure is illustrated in further details by the following non-limiting examples.

### Example 1: Preparation of 3D-Cell Culture Gel

1. Hydrogel preparation. 500 mg Methacrylated Gelatin (Advanced Biomatrix) was reconstituted in 5 mL of suitable media supplemented with 10% FBS. The hydrogel was warmed in 37° C. bath until complete dissolution. 0.5% of the photoinitiator lithium phenyl-2,4,6-trimethylbenzoylphosphinate (LAP) was added and mixed until a homogeneous solution was obtained. The mixture was kept warmed and in the dark to prevent polymerization.
2. Cell suspension. Cells were washed with 1×PBS and 0.25% trypsin was added to detach the cells from the petri dish, following by incubation at 37° C. Trypsin was inactivated by addition of media supplemented with 10% FBS. Cells were collected by centrifugation.
3. The cells were mixed with the hydrogel solution (5 million cells per 1 mL of hydrogel solution)
4. The cell/hydrogel suspension was poured into the mold according to the present disclosure.
5. The filled mold was exposed to 405 nm light for a time sufficient to induce polymerization of the hydrogel (during 1-1.5 min or until the gel whitens).
6. The two parts of the mold were separated, with the gel remaining in the upper part.
7. Culturing. The upper part containing the gel was inverted over a petri dish. The gel was pushed into the petri dish using a pipette tip inserted into the hole located in one of the corners. Media was added to cover the gel and the petri dish was incubated at 37° C., 5% CO₂.

### Example 2: Viability Results

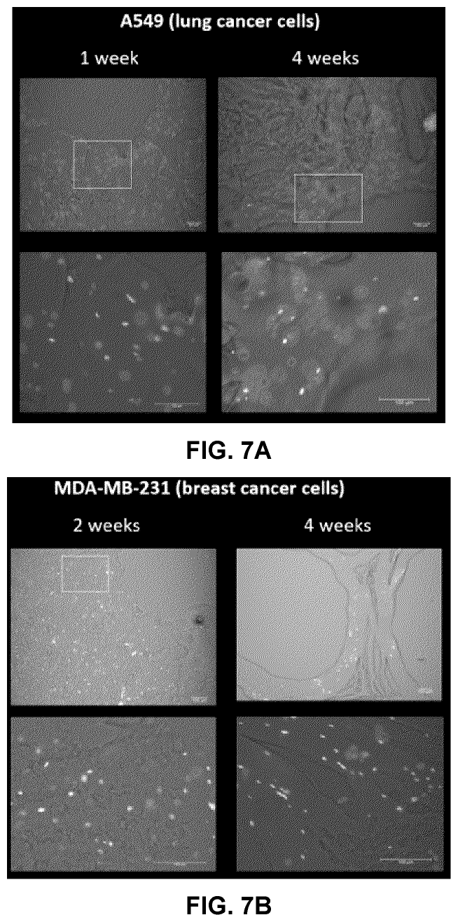

FIGS. 7A and 7B show the results of viability studies of a 3D-hydrogel containing A549 (lung cancer, FIG. 7A) and MDA-MB-231 (breast cancer, FIG. 7B) cells with a combination of fibroblasts (MRC5) and endothelial cells (HUVEC). Cell viability ranged from 60 to 70% measured after 1 week, 2 weeks and 4 weeks in culture.

### Example 3: Permeability of the 3D-Hydrogel

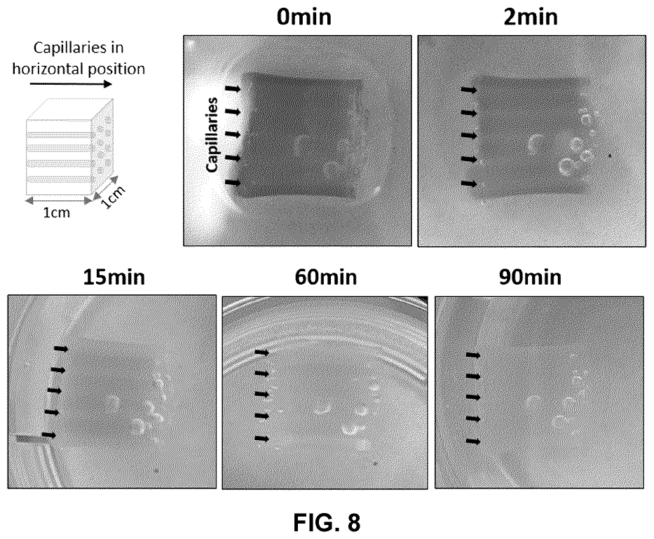

It was next tested whether 3D gels assembled with the devices and methods described herein are permeable. The 3D gel was formed and polymerized in the mold using collagen and cell culture media (giving a pink color to the gel). The 3D gel was extracted from the mold. Then, it was deposited in a petri dish filled with uncolored saline solution (PBS) and without agitation for 1 h30 at room temperature. The 3D gel was positioned in a way where capillaries are in a horizontal position, to favor saline diffusion inside the 3D gel. It was noticed that after 2 minutes, a discoloration of the interior of the gel at the position of the capillaries (marked by arrows in FIG. 8). After 15, 60 and 90 minutes, the discoloration increased rendering the gel fully transparent, suggesting that the component of the cell culture media (producing the pink color) could exit rapidly from the gel. This data suggests that the 3D gel with the capillaries is permeable and allows the passive diffusion of substances from the exterior to the inside of the gel (clear color saline solution) and from the inside of the gel to the outside of the gel (cell culture media).

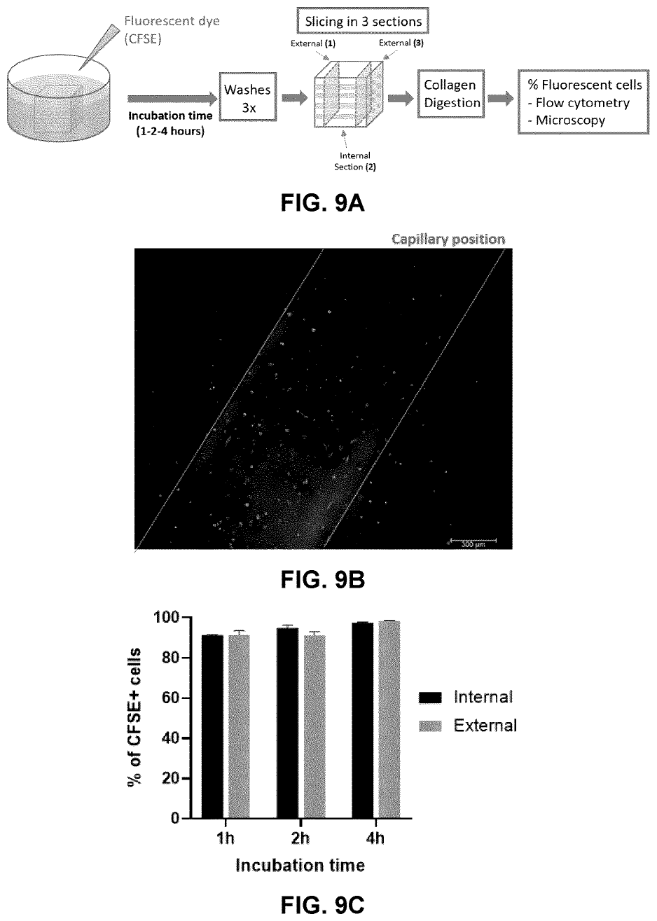

Additional assays were performed to demonstrate that the 3D gels assembled with the devices and methods described herein allow the rapid and uniform diffusion of an agent through the capillaries of the gel. A 3D gel containing collagen and a human cancer cell line (as a cell recipient) was assembled and placed into a petri dish containing cell culture media. Then, a cell-permeable fluorescent dye (5(6)-Carboxyfluorescein diacetate N-succinimidyl ester, CFSE) was added to the media for 1, 2 and 4 hours at 37° C. in an incubator. At each time point, the 3D gel was washed 3 times with media without the fluorescent dye to remove unspecific signal of the dye bound to the gel. Then, the 3D gel was cut in 3 sections to analyze separately the internal and external sections of the gel (FIG. 9A).

Using fluorescent microscopy (20× magnification), a section of a capillary after 1 h of incubation with the cell-permeable fluorescent dye (FIG. 9B) was imaged. The capillary position is marked by 2 parallel lines. Fluorescent cells (dots) were observed in the capillary and near the capillary. This suggest that cells are rapidly marked by the cell-permeable and that the agent can diffuse readily within the gel through the capillaries. Then, the 3D gel sections (external and internal) were digested by the enzymatic action of collagenase to obtain a suspension of human cancer cells. The fluorescent status of the cells in suspension was quantified by flow cytometry (FIG. 9C). The data demonstrate that the vast majority of human cancer cells (about 90%) within the 3D gel were rapidly stained within 1 h by the cell-permeable fluorescent dye. The staining was similar after 4 hours suggesting the diffusion of the agent can occur rapidly at least within 1 h, regardless of whether the position of the cell in the 3D gel, as the percentage of CFSE⁺ cells was similar between the internal and external sections of the gel.

Altogether, the data support the fact that 3D gels assembled with the devices and methods described herein are permeable to agents allowing a rapid and uniform diffusion of molecules.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

1. A 3D-cell-culture preparation device comprising:
a pronged component comprising a plurality of outwards prongs, the plurality of prongs being spatially distributed in accordance with a target artificial vasculature; and
a receiver component defining a 3D-cell-culture cavity, wherein when the receiver component is positioned onto the pronged component, the plurality of prongs protrude into, optionally through, the 3D-cell-culture cavity,
wherein the device is configured to receive a polymerizable liquid in the 3D-cell-culture cavity, optionally wherein an opening is defined to receive the liquid toward the 3D cell culture cavity and the 3D-cell culture-cavity is stanched to the liquid when the receiver component is operably positioned over the pronged component.

2. The device of claim 1, wherein the receiver component comprises a depression forming the 3D cell-culture cavity and the plurality of prongs protrudes into the 3D cell-culture cavity without protruding through the receiver component and wherein the receiver component comprises one or more holes to receive the polymerizable liquid in the 3D-cell-culture cavity, optionally the one or more holes is located at one or more of the corners of the base of the receiver component.

3-4. (canceled)

5. The device of claim 1, wherein the receiver component comprises a hole in a base thereof for each prong of the plurality of prongs.

6-7. (canceled)

8. The device of claim 1, further comprising a holder portion configured to facilitate separation of the receiver component and pronged component.

9. The device of claim 1, wherein the 3D-cell-culture cavity defines a volume to receive between 0.5 cubic centimeter and 1.5 cubic centimeters of the liquid; and/or wherein:
the spatial distribution leaves at least 0.4 mm between each of the plurality of prongs; and
the plurality of prongs occupies between 10% and 25% of a culture volume of the 3D cell-culture cavity, the culture volume being defined by a volume of liquid received in the 3D cell-culture cavity.

10. The device of claim 1, wherein the plurality of prongs are shaped as one or more of a cylinder, a polygonal base prism, an elliptical prism, a histologically-inspired prism and an enamel-inspired prism.

11. A method for preparing a 3D cell-culture, the method comprising:
(a) pouring a polymerizable liquid, optionally comprising cells, into the device of claim 1;
(b) exposing the device to solidification conditions of the polymerizable liquid thereby obtaining the 3D cell-culture embedding the target artificial vasculature; and optionally
(c) separating the pronged component and the receiver component of the device once the polymerizable liquid has solidified; and
(d) optionally, wherein the base of the receiver component comprises one or more holes located at one or more of corners and using one of the one or more holes to push the solidified gel out of the 3D-cell-culture cavity.

12-19. (canceled)

20. A system for preparing a 3D-cell-culture, the system comprising:
(a) the device of claim 1;
(b) a pouring module configured to controllably pour a polymerizable liquid into the device; and
(c) an exposition module configured to expose the device to solidification conditions of the liquid thereby obtaining the 3D cell culture embedding the target artificial vasculature.

21-26. (canceled)
